(19) United States
(12) Patent Application Publication
(10) Pub. No.: US 2021/0363591 A1
(43) Pub. Date: Nov. 25, 2021

(54) MAPK1 MUTATIONS AND DRUG SENSITIVITY

(71) Applicant: The Chinese University of Hong Kong, Shatin (CN)

(72) Inventors: Vivian Wai Yan Lui, Kowloon (CN); Hoi Lam Ngan, Kowloon (CN)

(21) Appl. No.: 17/193,615

(22) Filed: Mar. 5, 2021

Related U.S. Application Data
(60) Provisional application No. 62/987,157, filed on Mar. 9, 2020.

(51) Int. Cl. C12Q 1/6886; C12Q 1/6806

(52) U.S. Cl. CPC C12Q 1/6886; C12Q 2600/156; C12Q 1/6806

(57) ABSTRACT

The present invention provides a method for predicting a cancer patient's responsiveness to anti-EGFR cancer drugs such as erlotinib based on the presence of one or more mutations in the MAPK1 genomic sequence. Related kits and therapeutic methods are also provided.

### RELATED APPLICATIONS

This application claims priority to U.S. Provisional Patent Application No. 62/987,157, filed Mar. 9, 2020, the contents of which are hereby incorporated by reference in the entirety for all purposes.

### REFERENCE TO SUBMISSION OF A SEQUENCE LISTING AS A TEXT FILE

The Sequence Listing written in file 080015-1232912-030310US_SL.txt created on May 21, 2021, 19,923 bytes, machine format IBM-PC, MS-Windows operating system, is hereby incorporated by reference in its entirety for all purposes.

### BACKGROUND OF THE INVENTION

Cancer is one of the leading causes of morbidity and mortality worldwide, with approximately 15 million new cases diagnosed in each year of this decade. The number of new cases is expected to rise by about 70% over the next 2 decades. The second leading cause of death globally, cancer was responsible for 8.8 million deaths in 2015. The cost for treating cancer is extremely high: for example, in the year of 2014, approximately 88 billion US dollars was spent for cancer treatment in this country alone.

Because of the significant prevalence of cancer and the vital importance of effective treatment for cancer patients' survival and quality of life, and also because of increasingly more available options in cancer therapies, there exists an urgent need for new and meaningful ways of assessing different treatment methods for their likelihood of success in treating cancer patients depending on the patients' individual genetic traits such that individual patients can receive the most appropriate therapy and maximize their chance of survival. This invention fulfills this and other related needs.

### BRIEF SUMMARY OF THE INVENTION

By targeted sequencing analysis of cancer patients' genomic sequence, the present inventors have identified mutations in the MAPK1 gene that indicate the patients' responsiveness to anti-cancer drugs used in cancer therapy. For instance, among recurrent head and neck squamous cell carcinoma (HNSCC) patients, two MAPK1 mutations, MAPK1p.D321N and p.R135K, have been identified as correlating with heightened responsiveness to cancer treatment using EGFR inhibitors such as erlotinib or ErbB family inhibitors.

As such, in the first aspect, the present invention provides a method for assessing the likelihood of a cancer patient responding to a cancer drug, i.e., the likelihood of a targeted therapy (EGFR inhibitors, or ErbB family inhibitors) being effective in treating the cancer the patient has been diagnosed with. The method is based on the detection of a genetic mutation at one or more residues within at least one segment, such as or the 81-84, 131-139, and 317-321 segments, or the 77-84, 131-139, 143-152, 241-250, and 317-325 segments of human MAPK1 protein (the amino acid sequence for which is set forth in SEQ ID NO:1), or at one or more of residues 81, 135, 148, 246, and 321 of SEQ ID NO:1, or at one or more of the conserved kinase interaction motif (KIM) domain residues, the mutation being insertion, deletion, nonsense mutation, and/or substitution, especially non-conservative substitution, as well as gene rearrangement. The method includes these steps: (a) obtaining genomic DNA from biological sample taken from the patient; (b) determining the nucleotide sequence of a portion of MAPK1 genomic sequence encoding at least one of the 77-84, 131-139, 143-152, 241-250, and 317-325 segments of SEQ ID NO:1; (c) detecting one or more mutations within the portion of MAPK1 genomic sequence encoding at least one of the 77-84, 131-139, 143-152, 241-250, and 317-325 segments of SEQ ID NO:1; and (d) determining the cancer patient as someone who is likely to achieve effective treatment with EGFR inhibitors/ErbB family inhibitors. For example, the relevant mutation(s) may be present in the 77-84; 135-139; 145-149; 243-248; and 317-325 segment of SEQ ID NO:1.

In some embodiments, the biological sample used in the claimed method is a cancer biopsy or a blood sample. In some embodiments, the patient is diagnosed with and being assessed for potential treatment for head and neck squamous cell carcinoma (HNSCC). In some embodiments, the cancer is stomach cancer, esophageal cancer, thyroid cancer, penile cancer, breast cancer, leukemia, lymphoma, skin cancer, cervical cancer, bladder cancer, colorectal cancer, gallbladder cancer, brain cancer, ovarian cancer, pancreatic cancer, kidney cancer, prostate cancer, lung cancer, endometrial cancer, or liver cancer. In some embodiments, step (b) of the method comprises determining the nucleotide sequence of the portion of MAPK1 genomic sequence encoding at least one of the 77-84, 135-139, and 317-325 segments of SEQ ID NO:1. In some embodiments, step (c) of the method comprises detecting one or more mutations at residue 81, 135, or 321 of SEQ ID NO:1. The one or more mutations may include at least one substitution (e.g., non-conservative substitution) at residue 81, 135, or 321 of SEQ ID NO:1, for example, mutation E81K, R135K or D321N in SEQ ID NO:1. In some embodiments, step (c) of the method comprises detecting one or more mutations at residue 148 or 246 of SEQ ID NO:1. In some embodiments, step (b) of the method further comprises determining nucleotide sequence of the portion of MAPK1 genomic sequence encoding a segment of SEQ ID NO:1 encompassing residue 322. For example, a mutation E322 may be further present in the MAPK1 genomic sequence.

In some embodiments, the method further includes, subsequent to step (d), a step of administering to the cancer patient a targeted therapy drug, especially an EGFR inhibitor. For example, the targeted therapy drug may comprise erlotinib. In some embodiments, step (b) of the method comprises an amplification reaction, such as a polymerase chain reaction (PCR). In some embodiments, step (b) comprises a polynucleotide sequencing reaction. In some embodiments, step (b) comprises a polynucleotide hybridization assay.

In some embodiments, the method further includes, subsequent to step (d), a step of administering to the cancer patient a targeted therapy drug, especially an EGFR inhibitor or an ErbB family inhibitor. For example, erlotinib belongs to one of the FDA approved EGFR inhibitors for cancer treatment. In some embodiments, step (b) of the method comprises an amplification reaction, such as a polymerase chain reaction (PCR). In some embodiments, step (b) comprises a polynucleotide sequencing reaction. In some embodiments, step (b) comprises a polynucleotide hybridization assay.

In a second aspect, the present invention provides a kit for assessing likelihood of effective targeted therapy in a cancer patient. The kit comprises (1) two oligonucleotide primers capable of specifically amplifying a portion of MAPK1 genomic sequence obtained from a biological sample taken from the cancer patient, wherein the portion of MAPK1 genomic sequence encodes at least one of the 81-84, 131-139, and 317-321 segments, or the 77-84, 131-139, 143-152, 241-250, and 317-325 segments of SEQ ID NO:1, or any segment of SEQ ID NO:1 encompassing residue 81, 135, 148, 246, or 321, or the KIM domain residues; and (2) an agent capable of determining nucleotide sequence of the portion of MAPK1 genomic sequence encoding at least one of the 81-321 segment, or the 81-84, 131-139, and 317-321 segments, or the 77-84, 131-139, 143-152, 241-250, and 317-325 segments of SEQ ID NO:1, or any segment of SEQ ID NO:1 encompassing residue 81, 135, 148, 246, or 321, or the KIM residues. For example, the relevant primers and reagents may be used for amplify and determine the nucleotide sequence of a portion of MAPK1 genomic sequence encoding at least one of the 77-84; 135-139; 145-149; 243-248; and 317-325 segment of SEQ ID NO:1. In some embodiments, the portion of MAPK1 genomic sequence to be analyzed encodes at least one of the 81-84, 135-139, and 317-321 segments of SEQ ID NO:1. In some embodiments, the biological sample is a cancer biopsy or a blood sample. In some embodiments, the cancer is head and neck squamous cell carcinoma (HNSCC). In some embodiments, the cancer is stomach cancer, esophageal cancer, thyroid cancer, penile cancer, breast cancer, leukemia, lymphoma, skin cancer, cervical cancer, bladder cancer, colorectal cancer, gallbladder cancer, brain cancer, ovarian cancer, pancreatic cancer, kidney cancer, prostate cancer, lung cancer, endometrial cancer, or liver cancer. Typically, the kit will further include an instruction manual.

In a third aspect, the present invention provides a method for treating a cancer patient. The method includes the step of administering to a cancer patient, whose genomic sequence has been analyzed and confirmed to comprise at least one mutation in a portion of MAPK1 genomic sequence encodes at least one of the 81-84, 131-139, and 317-321 segments, or the 77-84, 131-139, 143-152, 241-250, and 317-325 segments of SEQ ID NO:1, or at one or more of residues 81, 135, 148, 246, and 321, or at one or more of the KIM domain residues, an effective amount of a targeted therapy drug such as an EGFR inhibitor. The mutation may be addition, deletion, nonsense mutation and/or substitution, especially non-conservative substitution, as well as gene rearrangement. For example, at least one mutation may be present in at least one of the 77-84, 135-139, 145-149, 243-248, and 317-325 segment of SEQ ID NO:1. In some embodiments, the method further includes, prior to the administering step, a step of selecting or identifying a cancer patient suitable for targeted therapy for treating cancer by analyzing the patient's genomic sequence and confirming the presence of at least one mutation in a portion of MAPK1 genomic sequence encoding at least one of the 77-84, 131-139, 143-152, 241-250, and 317-325 segments of SEQ ID NO:1. In some embodiments, the patient has at least one mutation in the portion of MAPK1 genomic sequence encoding at least one of the 81-84, 135-139, and 317-321 segments of SEQ ID NO:1. In some embodiments, the patient has at least one mutation, especially substitution (e.g., non-conservative substitution), at residues 81, 135, and 321 of SEQ ID NO:1, such as having at least one substitution of E81K, R135K or D321N in SEQ ID NO:1. In some embodiments, the patient has at least one mutation at residue 148 or 246 of SEQ ID NO:1. In some embodiments, the cancer is head and neck squamous cell carcinoma (HNSCC). In some embodiments, the cancer is stomach cancer, esophageal cancer, thyroid cancer, penile cancer, breast cancer, leukemia, lymphoma, skin cancer, cervical cancer, bladder cancer, colorectal cancer, gallbladder cancer, brain cancer, ovarian cancer, pancreatic cancer, kidney cancer, prostate cancer, lung cancer, endometrial cancer, or liver cancer. In some embodiments, the targeted therapy drug is an EGFR inhibitor such as erlotinib, or an ErbB family inhibitor.

### BRIEF DESCRIPTION OF THE DRAWINGS

FIG. 1A: Table showing HNSCC cases with somatic MAPK1 mutations in the US-TCGA-HNSCC Provisional cohort (N=512 tumors) and the Asian HK-HNSCC cohort (N=105 tumors). FIG. 1B: Mapping of mutation sites of the MAPK1 gene based on the pan-cancer data from TCGA^{9,10} and the COSMIC¹¹ database. Each mutational event is presented by one triangle symbol. Color annotation of cancer types are shown at the bottom. FIG. 1C: Conserved regions of the MAPK1 (ERK2) proteins across species around amino acid positions p.D321 and p.R135 are shown. The amino acid residues of the KIM docking site are indicated by red arrows. FIG. 1D: The X-ray crystallography structure of the human MAPK1(ERK2) protein (locked with the ATP competitive inhibitor 5-Iodotubercidin and the allosteric inhibitor peptide-type ERK2 inhibitor; PDB ID: 5AX3¹³; MMDB ID: 136379¹⁴). Amino acid residues R135, D321 and E322 are highlighted in red, blue and green, respectively. Residue R135 is 9.0 Å away from E322 and 11.3 away from D321. The peptide sequence of the KIM-domain is highlighted and labeled in yellow. FIG. 1E: The same X-ray crystallography structure of MAPK1 protein showing the peptide sequence of the ATP binding domain highlighted in yellow, and the ATP molecule shown in grey color. FIG. 1F: Driver activity assay, by MTT assay, of FaDu cells that ectopically expressed MAPK1 WT, MAPK1 p.D321N and MAPK1 p.R135K mutants. Cells were seeded on a 48-well plate at a density of 1.2×10⁴ cell/well with DMEM and 5% FBS. MTT assay were conducted at 96 hrs after seeding. A cumulative graph of 3 independent repeats is shown (total N≥14 wells). Driver activity was normalized against MAPK1 WT. The MAPK1 p.D321N is a driver for FaDu cell growth (P<0.0001; 88.65%±1.262 SEM), while the MAPK1 p.R135K moderately suppresses cell growth (P<0.0001; 122.3% 4.060 SEM). FIG. 1G: Western blotting showing the level of p-EGFR(Y1173), t-EGFR, p-MAPK and t-MAPK in FaDu cells expressing MAPK1 WT, MAPK1 p.R135K and MAPK1 p.D321N mutants, respectively. The p-EGFR and total EGFR levels were normalized to actin, and shown as bar graphs. Three independent repeats were performed and all repeats showed similar results.

FIG. 2A: Fractional tumor growth curves of in vivo tumor expressing MAPK1 WT, MAPK1 p.D321N or MAPK1 p.R135K (mean tumor sizes with SE). FaDu cells expressing MAPK1 WT, MAPK1 p.D321N and MAPK1 p.R135K respectively were inoculated into nude mice subcutaneously (8×10⁵ cells per inoculation). Mice with tumor expressing respective MAPK1 WT/mutations were randomized into erlotinib (erlotinib dissolved in 10% 2-Hydroxypropyl-beta-cyclodextrin (HP-j-CD) or vehicle (10% HP-β-CD) treatment groups (N=8 tumors per group). Treatment started when tumors were palpable and reached the size ˜3×3-4×4 mm². Erlotinib or the vehicle control were administered by oral gavage (50 mg/kg erlotinib or the corresponding vehicle amount) for 5 consecutive days as indicated by the arrows on the X-axis. FIG. 2B: IHC staining showing membranous p-EGFR expression of these xenografts post erlotinib/vehicle treatments (N>3 per group). 100 μm scale bars were shown. FIG. 2C: IHC staining showing corresponding pan-cytokeratin expression in these tumors post erlotinib/vehicle treatment (N>3 per group). 50 μm scale bars were shown.

### DEFINITIONS

The term “MAPK1,” as used herein, refers to mitogen-activated protein kinase 1, also known as extracellular signal-regulated kinase 2 (ERK2). It is a member of the MAP kinase or ERK family. The genomic DNA sequence for human MAPK1 is set forth in GenBank Accession No. NM_002745.5 and provided herein as SEQ ID NO:2, which translates to a coding sequence for a 360-amino acid MAPK1 protein (set forth in GenBank Accession No. NP_002736.3, provided herein as SEQ ID NO:1).

In this disclosure the term “or” is generally employed in its sense including “and/or” unless the content clearly dictates otherwise.

In this disclosure the term “biological sample” includes sections of tissues such as biopsy and autopsy samples, and frozen sections taken for histologic purposes, or processed forms of any of such samples. Biological samples include blood and blood fractions or products (e.g., serum, plasma, platelets, red blood cells, white blood cells, all blood cells, and the like), sputum or saliva, lymph and tongue tissue, buccal cells, cultured cells, e.g., primary cultures, explants, and transformed cells, stool, urine, or tumor biopsy tissue etc. A biological sample is typically obtained from a eukaryotic organism, which may be a mammal, may be a primate and may be a human subject.

In this disclosure the term “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (e.g., tongue, oral cavity, prostate, kidney, bladder, lymph node, liver, bone marrow, blood cells, etc.) among other factors. Representative biopsy techniques include, but are not limited to, excisional biopsy, incisional biopsy, needle biopsy, surgical biopsy, and bone marrow biopsy. A wide range of biopsy techniques are well known to those skilled in the art who will choose between them and implement them with minimal experimentation.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

In this application, the terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid.

There are various known methods in the art that permit the incorporation of an unnatural amino acid derivative or analog into a polypeptide chain in a site-specific manner, see, e.g., WO 02/086075.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another (conversely, substitution between any two amino acids belonging to two different groups is referred to as “non-conservative substitution”):

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins, W. H. Freeman and Co., N. Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

A “polynucleotide hybridization method” as used herein refers to a method for detecting the presence and/or quantity of a pre-determined polynucleotide sequence based on its ability to form Watson-Crick base-pairing, under appropriate hybridization conditions, with a polynucleotide probe of a known sequence. Examples of such hybridization methods include Southern blot, Northern blot, and in situ hybridization.

“Primers” as used herein refer to oligonucleotides that can be used in an amplification method, such as a polymerase chain reaction (PCR), to amplify a nucleotide sequence based on the polynucleotide sequence corresponding to a gene of interest, e.g., the genomic sequence for human MAPK1 or a portion thereof. Typically at least one of the PCR primers for amplification of a polynucleotide sequence is sequence-specific for that polynucleotide sequence. The exact length of the primer will depend upon many factors, including temperature, source of the primer, and the method used. For example, for diagnostic and prognostic applications, depending on the complexity of the target sequence, the oligonucleotide primer typically contains at least 10, or 15, or 20, or 25 or more nucleotides, although it may contain fewer nucleotides or more nucleotides. The factors involved in determining the appropriate length of primer are readily known to one of ordinary skill in the art. In this disclosure the term “primer pair” means a pair of primers that hybridize to opposite strands a target DNA molecule or to regions of the target DNA which flank a nucleotide sequence to be amplified. In this disclosure the term “primer site”, means the area of the target DNA or other nucleic acid to which a primer hybridizes.

A “label,” “detectable label,” or “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins that can be made detectable, e.g., by incorporating a radioactive component into the peptide or used to detect antibodies specifically reactive with the peptide. Typically a detectable label is attached to a probe or a molecule with defined binding characteristics (e.g., a polypeptide with a known binding specificity or a polynucleotide), so as to allow the presence of the probe (and therefore its binding target) to be readily detectable.

The term “treat” or “treating,” as used in this application, describes to an act that leads to the elimination, reduction, alleviation, reversal, or prevention or delay of onset or recurrence of any symptom of a relevant condition. In other words, “treating” a condition encompasses both therapeutic and prophylactic intervention against the condition.

The term “effective amount” as used herein refers to an amount of a given substance that is sufficient in quantity to produce a desired effect. For example, an effective amount of an anti-cancer agent is the amount of said agent to achieve a detectable effect, such as reduced, reversed, eliminated, or prevented cancer symptom(s) or delayed of the onset of such symptom(s) in a patient who has been given the agent for therapeutic purposes. An amount adequate to accomplish this is defined as the “therapeutically effective dose.” The dosing range varies with the nature of the therapeutic agent being administered and other factors such as the route of administration and the severity of a patient's condition.

The term “subject” or “subject in need of treatment,” as used herein, includes individuals who seek medical attention due to risk of, or actual suffering from, cancer. Subjects also include individuals currently undergoing therapy that seek manipulation of the therapeutic regimen. Subjects or individuals in need of treatment include those that demonstrate symptoms of cancer or are at risk of suffering from cancer or its symptoms. For example, a subject in need of treatment includes individuals with a diagnosis of cancer, those with a genetic predisposition or family history for cancer, those that have suffered relevant symptoms in the past, those that have been exposed to a triggering substance or event, as well as those suffering from chronic or acute symptoms of the condition. A “subject in need of treatment” may be at any age of life.

“Inhibitors,” “activators,” and “modulators” of a target protein are used to refer to inhibitory, activating, or modulating molecules, respectively, identified using in vitro and in vivo assays for the target protein binding or signaling, e.g., ligands, agonists, antagonists, and their homologs and mimetics. The term “modulator” includes inhibitors and activators. Inhibitors are agents that, e.g., partially or totally block carbohydrate binding, decrease, prevent, delay activation, inactivate, desensitize, or down regulate the activity of the target protein. In some cases, the inhibitor directly or indirectly binds to the target protein, such as a neutralizing antibody. Inhibitors, as used herein, are synonymous with inactivators and antagonists. Activators are agents that, e.g., stimulate, increase, facilitate, enhance activation, sensitize or up regulate the activity of target protein. Modulators include ligands or binding partners for the target protein, including modifications of naturally-occurring ligands and synthetically-designed ligands, antibodies and antibody fragments, antagonists, agonists, small molecules including carbohydrate-containing molecules, siRNAs, RNA aptamers, and the like.

The epidermal growth factor receptor (EGFR, also known as ErbB-1/HER1 in human) is a transmembrane protein that is a receptor for members of the epidermal growth factor family (EGF family) of extracellular protein ligands. EGFR is a member of the ErbB family of receptors, a subfamily of four closely related receptor tyrosine kinases: EGFR (ErbB-1), HER2/neu (ErbB-2), Her 3 (ErbB-3) and Her 4 (ErbB-4). In many cancer types, mutations or copy number alterations affecting EGFR expression or activity may be present. EGFR is activated upon binding of its specific ligands and undergoes a transition from an inactive monomeric form to an active homodimer, which in turn stimulates its intrinsic intracellular protein-tyrosine kinase activity. As a result, autophosphorylation of several tyrosine (Y) residues (such as Y992, Y1045, Y1068, Y1148 and Y1173) in the C-terminal domain of EGFR occurs and triggers downstream signaling. Thus, an EGFR inhibitor is a compound that is capable of suppressing EGFR activity or downstream signaling either directly (e.g., via direct interaction with EGFR itself) or indirectly and is therefore useful in anti-EGFR cancer therapy, especially in cancer types involving aberrant activation or increased expression of EGFR.

### DETAILED DESCRIPTION OF THE INVENTION

### I. Introduction

The present inventors discovered for the first time that presence of genetic mutation or mutations within certain segments of the MAPK1 gene is indicative of likely effectiveness of cancer therapy by cancer drugs such as epidermal growth factor receptor (EGFR) inhibitors or ErbB family inhibitors. The worldwide incident rate of Head and Neck Cancer (HNC) is estimated to reach 0.7 million cases in 2019. However, for this group of patients, the 5-year survival rate is about 50% only and the median overall survival (OS) for recurrent HNC is even as short as ˜7 months. Facing the poor OS rate, the choice of FDA-approved targeted therapy is very limited. In 2006, cetuximab, an EGFR inhibitor, was approved for recurrent/metastatic HNC without genomic guidance, but its objective response rate is only moderate.

EGFR is upregulated or activated in HNC as well as many human cancers. Yet, the question of how to target EGFR signaling in cancer therapy in a precision manner so as to achieve good antitumor response remains unanswered for these cancers. Except for EGFR-mutated lung cancer, where EGFR-activating mutations can drive EGFR phosphorylation, thus high drug sensitivity towards EGFR inhibitors have been clinically implemented for the treatment of EGFR-mutated lung cancer as a precision therapy. In their studies, the present inventors discovered that MAPK1 p.D321N mutation resides in the conserved kinase interaction motif (KIM) domain of MAPK1 and can activate EGFR signaling in HNC both in vitro and in vivo, and conferred drug sensitivity to an EGFR inhibitor. It was also discovered that another mutation, MAPK1 p.R135K, where the R135 amino acid residue of MAPK1 protein within the KIM domain and located in 3D close proximity to D321, is also able to drive EGFR activation, and some level of sensitivity towards EGFR inhibitor in vivo. Previously, a U.S. HNC patient with tumor haboring an MAPK1 p.E322K mutation, which happened just next to the KIM domain, had also shown exceptional clinical response to EGFR inhibitor (Van Allen and Lui et al., Genomic correlate of exceptional erlotinib response in head and neck squamous cell carcinoma. JAMA Oncology. (2015). May 1; 1(2):238-44). Mutations of all these sites can activate EGFR activation in HNSCC cells. Thus, these drug-sensitizing MAPK1 mutations have the ability to activate EGFR signaling in HNC, and potentially in other human cancer, thus making them sensitive to EGFR inhibitors used in targeted cancer therapy. Thus, these newly identified mutations in MAPK1 protein, especially in the KIM domain, can serve as drug sensitivity biomarkers guiding precision use of EGFR inhibitors for the treatment of HNC and other cancers in patients.

### II. General Methodology

Practicing this invention utilizes routine techniques in the field of molecular biology. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255: 137-149 (1983).

The sequence of interest used in this invention, e.g., the polynucleotide sequence of the human MAPK1 gene, and synthetic oligonucleotides (e.g., primers) can be verified using, e.g., the chain termination method for sequencing double-stranded templates of Wallace et al., Gene 16: 21-26 (1981).

### III. Acquisition of Tissue Samples and Analysis of Genomic DNA

The present invention relates to analyzing the nucleotide sequence of MAPK1 genomic DNA found in a person's tissue sample as a means to detect the presence of genetic mutations relevant to drug sensitivity. Thus, the first steps of practicing this invention are to obtain a tissue sample from a test subject and extract genomic DNA from the sample, with the tissue sample generally corresponding to the organ/tissue for which cancer status is to be assessed (e.g., a cancer tissue sample such as biopsy is analyzed to detecting the presence of pertinent genetic mutation(s) in head and neck cancer patients for the purpose of identifying somatic mutations or even the presence of germline mutation that should be theoretically detected in the tumor tissue of a patient, or a blood sample is analyzed to detect the presence of pertinent molecular mutation(s) in the blood cells, such as leukemia or lymphoma).

A. Acquisition and Preparation of Tissue Samples

A tissue sample is obtained from a person to be tested or assessed for future cancer therapy using a method of the present invention. Collection of various types of tissue sample, e.g., solid tissue sample or blood sample, from an individual is performed in accordance with the standard protocol hospitals or clinics generally follow, such as during a biopsy or blood draw. An appropriate amount of tissue sample is collected from a chosen organ or anatomic site and may be stored according to standard procedures prior to further preparation.

The analysis of MAPK1 genomic DNA found in a patient's tissue sample according to the present invention may be performed using routine methodologies. The methods for preparing tissue samples for nucleic acid extraction are well known among those of skill in the art. For example, a subject's solid or liquid tissue sample should be first treated to disrupt cellular membrane so as to release nucleic acids contained within the cells.

B. Analysis of MAPK1 Genomic Sequence

The presence of genetic mutation(s) in a segment of MAPK1 genomic sequence (such as the KIM domain) pairs is investigated to provide indication as to whether a cancer patient is likely to benefit from targeted therapy, especially where an EGFR inhibitor or an ErbB family inhibitor is used for treating cancer.

Typically a segment of the MAPK1 genomic sequence, such as one that encodes one or more of the segments of the MAPK1 protein, for example, the 77-84, 131-139, 143-152, 241-250, and 317-325 segments of SEQ ID NO:1, or the 81-84, 133-137, 146-150, 244-248, and 317-321 segments of SEQ ID NO:1, such that any point mutation or mutations, including insertion, deletion, or substitution that would result in changes in the encoded amino acid sequence (e.g., resulting in non-conservative substitution(s) in the amino acid sequence), in these segments can be detected and ascertained in the resulting nucleotide sequence. More specifically, the potential point mutations at one or more of residues 81, 135, 148, 246, and 321 of MAPK1 protein (SEQ ID NO:1), or one or more of the KIM domain residues of MAPK1 protein (SEQ ID NO:1) may be detected. Further, MAPK1 genomic sequence outside of the KIM domain may also be analyzed, since certain residues, such as residue 322 of SEQ ID NO:1, have been previously revealed to be relevant to drug-sensitivity in a cancer patient.

1. DNA Extraction

Methods for extracting DNA from a biological sample are well-known and routinely practiced in the art of molecular biology, see, e.g., Sambrook and Russell, supra. RNA contamination should be eliminated to avoid interference with DNA analysis.

2. Optional Amplification and Sequence Analysis

Following the extraction of genomic DNA from a patient sample, the genomic DNA is then subjected to sequence-based analysis, such that the presence of relevant mutation(s) in the MAPK1 genomic sequence may be detected. An amplification reaction is optional prior to the sequence analysis. A variety of polynucleotide amplification methods are well established and frequently used in research. For instance, the general methods of polymerase chain reaction (PCR) for polynucleotide sequence amplification are well-known in the art and are thus not described in detail herein. For a review of PCR methods, protocols, and principles in designing primers, see, e.g., Innis, et al., PCR Protocols: A Guide to Methods and Applications, Academic Press, Inc. N.Y., 1990. PCR reagents and protocols are also available from commercial vendors, such as Roche Molecular Systems.

Although PCR amplification is typically used in practicing the present invention, one of skill in the art will recognize that amplification of the relevant genomic sequence may be accomplished by any known method, such as the ligase chain reaction (LCR), transcription-mediated amplification, and self-sustained sequence replication or nucleic acid sequence-based amplification (NASBA), each of which provides sufficient amplification.

Techniques for polynucleotide sequence determination are also well established and widely practiced in the relevant research field. For instance, the basic principles and general techniques for polynucleotide sequencing are described in various research reports and treatises on molecular biology and recombinant genetics, such as Wallace et al., supra; Sambrook and Russell, supra, and Ausubel et al., supra. DNA sequencing methods routinely practiced in research laboratories, either manual or automated, can be used for practicing the present invention. Additional means suitable for detecting mutation(s) in a polynucleotide sequence for practicing the methods of the present invention include but are not limited to mass spectrometry, primer extension, polynucleotide hybridization, real-time PCR, melting curve analysis, high resolution melting analysis, heteroduplex analysis, pyrosequencing, and electrophoresis.

### IV. Treatment of Cancer

By illustrating the correlation of mutation(s) present in certain segments of the MAPK1 protein found in a cancer patient's tissue sample and the patient's drug sensitivity to cancer treatment, especially to EGFR inhibitor-based treatment, the present invention further provides a means for personalized cancer therapy for patients diagnosed with cancer, including head and neck cancer: by way of detecting the presence or absence of mutation or mutations in the MAPK1 protein relevant to drug sensitivity, the present invention allows for a treating physician to decide whether a particular patient should or should not undergo targeted therapy comprising the administration of one or more cancer drugs, for example, EGFR inhibitors such as erlotinib, or ErbB family inhibitors. More specifically, when one or more mutations (which may include additions, deletions, or substitutions) are detected at one or more residues within one or more segments of the MAPK1 protein, especially the KIM domain, the cancer patient is deemed to likely achieve successful treatment by cancer drugs such as EGFR inhibitor (e.g., erlotinib) and therefore is selected to receive administration of an effective amount of the drug. The MAPK1 protein segments that potentially harbor relevant mutations include the 77-84, 131-139, 143-152, 241-250, and 317-325 segments of SEQ ID NO:1, or the 81-84, 133-137, 146-150, 244-248, and 317-321 segments of SEQ ID NO:1, or the KIM residues within SEQ ID NO:1. The point mutations relevant to drug sensitivity include those at residues 81, 135, 148, 246, and 321, or at the KIM domain residues the of MAPK1 protein may be detected. Moreover, the presence of genomic mutations in the amino acid sequence segment outside of the KIM domain (such as residue 322 of SEQ ID NO:1) may also be analyzed to provide further supportive information in guiding the treating physician's decision on electing the proper treatment method for individual patients. Although different types of mutations are possible, the often-observed mutations are substitutions, especially non-conservative substitutions, of amino acids. On the other hand, when no mutation is detected at any of the specified residues within any of the specified segments of the MAPK1 protein, especially the KIM domain, the cancer patient may be less likely to achieve successful treatment by cancer drugs specifically targeting EGFR. These patients may be recommended for treatment using alternative methods including but not limited to surgery, radiation therapy, and non-specific chemotherapy (for example, using an anti-proliferative agent not specifically targeting EGFR, e.g., actinomycin D, bleomycin, cisplatin, doxorubicin, gemcitabine, hycamtin, mitomycin C, pentostatin, raloxifene, and vincristine).

Exemplary anti-cancer drugs suitable for use in the treat method of this invention include but are not limited to EGFR inhibitors or other ErbB family inhibitors, such as erlotinib, gefitinib, cetuximab, dacomitinib, lapatinib, afatinib, trastuzumab, pertuzumab, necitumumab, osimertinib, neratinib, panitumumab, and vandetinib, varlitinib, canertinib, pelitinib, sapitinib, and poziotinib.

### V. Kits and Devices

The invention provides compositions and kits for practicing the methods described herein to assess the presence of relevant genetic mutation(s) in MAPK1 genomic DNA (e.g., in the segment of MAPK1 genomic sequence encoding at least one segment of in SEQ ID NO:1, such as the 77-84, 131-139, 143-152, 241-250, and 317-325 segments of SEQ ID NO:1, or the 81-84, 133-137, 146-150, 244-248, and 317-321 segments of SEQ ID NO:1, encompassing point mutations at residue 81, 135, 148, 246, or 321, or any one or more of the KIM domain residues of the MAPK1 protein) in the genomic DNA obtained in a biological sample taken from a cancer patient, which then can be used for determining the likelihood of successful treatment of the cancer, such as by an EGFR inhibitor (e.g., erlotinib).

Kits for carrying out assays for determining MAPK1 genomic sequence and therefore assessing therapeutic efficacy of targeted cancer therapy in a cancer patient typically include a pair of oligonucleotide primers useful for specific hybridization with at least one segment of the MAPK1 coding sequence or its complementary sequence so as to allow amplification of a genomic sequence segment encoding at least one of the 77-84, 131-139, 143-152, 241-250, and 317-325 segments of SEQ ID NO:1, or the 81-84, 133-137, 146-150, 244-248, and 317-321 segments of SEQ ID NO:1, or any other segments encompassing one or more of residues 81, 135, 148, 246, and 321 of SEQ ID NO:1 or one or more of the KIM domain residues. Also included in the kit is a reagent that is capable of determining nucleotide sequence of the portion of MAPK1 genomic sequence encoding at least one of the 77-84, 131-139, 143-152, 241-250, and 317-325 segments of SEQ ID NO:1, or the 81-84, 133-137, 146-150, 244-248, and 317-321 segments of SEQ ID NO:1, or any other segments encompassing one or more of residues 81, 135, 148, 246, and 321 of SEQ ID NO:1 or one or more of the KIM domain residues. For example, the kit contains primers and reagent for amplifying/analyzing/determining the portion of MAPK1 genomic sequence encodes at least one of the 81-84, 135-139, and 317-321 segments of SEQ ID NO:1.

Optionally, an oligonucleotide probe is included in the kit for the purpose of detecting one or more mutations, for example, within a portion of MAPK1 genomic sequence encoding at least one of the 77-84, 131-139, 143-152, 241-250, and 317-325 segments of SEQ ID NO:1, or the 81-84, 133-137, 146-150, 244-248, and 317-321 segments of SEQ ID NO:1, or any other segments encompassing one or more of residues 81, 135, 148, 246, and 321 of SEQ ID NO:1, or at one or more of residues 81, 135, 148, 246, and 321 of SEQ ID NO:1, or at one or more of the KIM domain residues. This oligonucleotide probe is labeled with a detectable moiety and specifically hybridizes to a polynucleotide sequence corresponding to the MAPK1 genomic sequence with or without one or more of the specific mutations. In some cases, the kits may include at least two or more different oligonucleotide probes that each specifically hybridizes to a distinct version of at least a portion of the MAPK1 genomic sequence containing no mutation or one or more different mutations, particularly after an amplification process such as by PCR. In addition, the kits of this invention may provide instruction manuals to guide users in analyzing test samples and assessing the prospect of effective cancer treatment in a test subject, a patient who has been diagnosed with cancer, such as head and neck cancer.

In a further aspect, the present invention can also be embodied in a device or a system comprising one or more such devices, which is capable of carrying out all or some of the method steps described herein. For instance, in some cases, the device or system performs the following steps upon receiving a tissue sample, e.g., a cancer biopsy or blood sample taken from a cancer patient being tested for detecting potential mutations in the MAPK1 genomic sequence relevant to efficacy of targeted cancer therapy: (a) determining in a biological sample taken from a cancer patient the nucleotide sequence of a portion of MAPK1 genomic sequence encoding at least one of the 77-84, 131-139, 143-152, 241-250, and 317-325 segments of SEQ ID NO:1, or the 81-84, 133-137, 146-150, 244-248, and 317-321 segments of SEQ ID NO:1, or any other segments encompassing one or more of residues 81, 135, 148, 246, and 321 of SEQ ID NO:1 or one or more of the KIM domain residues of the MAPK1 protein; (b) detecting the presence or absence of one or more mutations in this portion of the MAPK1 genomic sequence, for example, mutation or mutations resulting addition, deletion, or substitution at one or more amino acid residues in these segments of the MAPK1 protein, such as residues 81, 135, 148, 246, and 321 or one or more of the KIM domain residues of SEQ ID NO:1; and (c) providing an output indicating whether the cancer patient is likely to benefit from cancer targeted therapy employing a cancer drug such as an EGFR inhibitor (for example, erlotinib) or ErbB family inhibitors, i.e., whether or not the patient is likely to respond to the cancer drug and achieve a successful treatment outcome in alleviating the patient's cancer condition. In other cases, the device or system of the invention performs the task of steps (b) and (c), after step (a) has been performed elsewhere and the genomic sequence information from step (a) has been entered into the device/system. Preferably, the device or system is partially or fully automated.

### Examples

The following examples are provided by way of illustration only and not by way of limitation. Those of skill in the art will readily recognize a variety of non-critical parameters that could be changed or modified to yield essentially the same or similar results.

### INTRODUCTION

Erlotinib is an FDA-approved agent for the treatment of epidermal growth factor receptor (EGFR)-mutated non-small cell lung cancer (NSCLC) and pancreatic cancer. Particularly for EGFR-mutated NSCLC, remarkable increase in progression-free survival was observed in Phase III trials^{1,2}. In polycythemia vera, some JAK2p.V617-mutated patients have demonstrated erlotinib sensitivity³. Recently, early clinical trial data in head and neck squamous cell carcinoma (HNSCC) revealed the presence of an EGFR-AS1 (c.2361G>A) synonymous mutation⁴, high baseline phospho-MAPK⁵ and MAPK1 p.E322K mutation⁶ as additional potential biomarkers for erlotinib sensitivity. As HNSCC lacks predictive biomarkers for drug responses, in-depth studies were conducted on MAPK1p.E322K, the mutation found in a complete erlotinib responder. Subsequent results revealed MAPK1p.E322K's ability to hyperactivate EGFR, which could confer erlotinib sensitivity in HNSCC^{6,7}.

HNSCC frequently recurs. Once recurred, patients have dismal survivals of only ˜7 months⁸. Here, by targeted sequencing, the present inventors have identified 2 MAPK1 somatic mutations, p.D321N and p.R135K, in 2 cases of primary-recurrent HNSCC from Hong Kong. The aim of this study is to determine if these two recurrence-associated MAPK1 mutations may also confer erlotinib sensitivity in HNSCC as reported for MAPK1p.E322K^{6,7}. The findings show that MAPK1p.D321N confers heightened sensitivity to erlotinib in vivo, while p.R135K's effect is moderate.

### Results:

### Potential High Rate of MAPK1 Mutations in Asian HNSCC

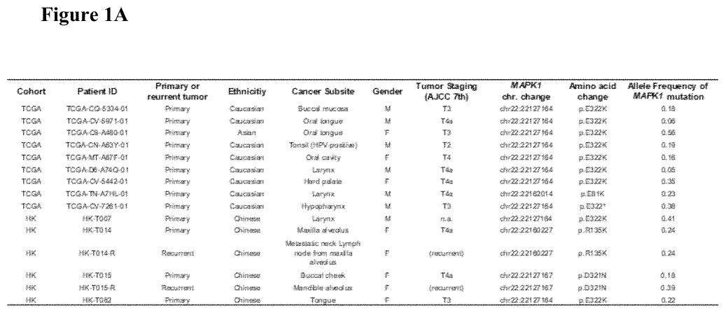

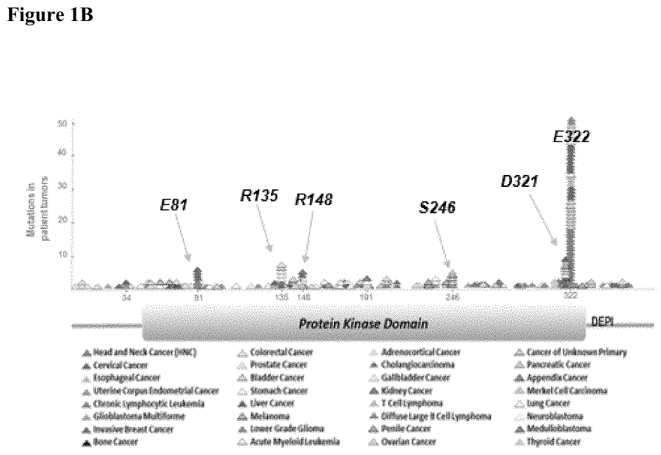

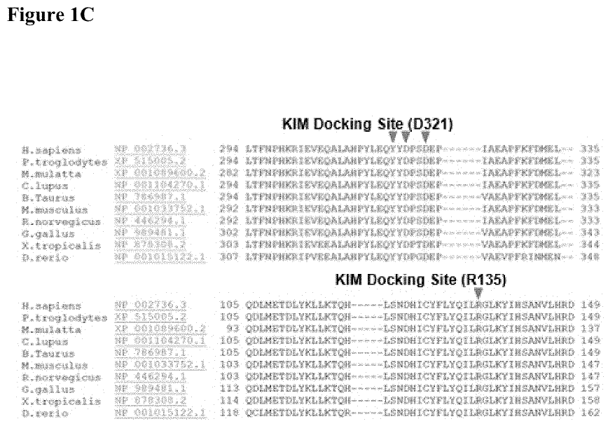

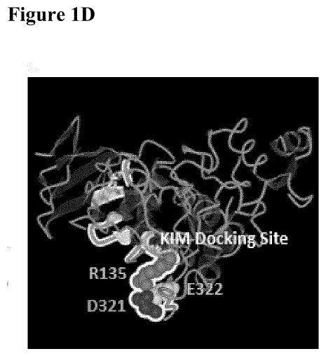

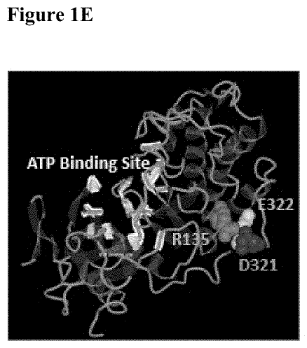

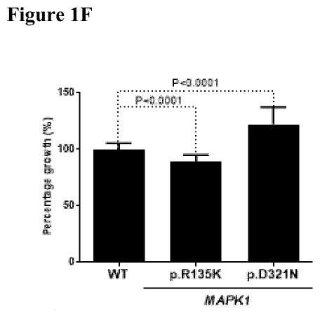

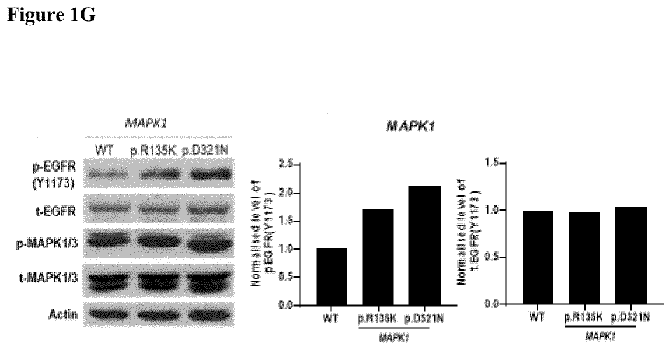

In 32 The Cancer Genome Atlas (TCGA) pan-cancers^{9,10}, the average MAPK1 mutation rate is 0.79% (86/10953 cases, 32 TCGA pan-cancers, as of August 2019). Notably, MAPK1 mutation rate in HNSCC appears to be relatively higher (1.8%; 9/512 cases) than that in the TCGA pan-cancers, and such HNSCC-associated MAPK1 mutations are almost all uniformly E322K or E322* mutations (FIG. 1a). Interestingly, a relatively diverse MAPK1 mutation pattern and a relatively higher mutation rate of MAPK1 (5.7%; 6/105 fresh frozen tumors from 103 unique individuals) were identified in our small Hong Kong HNSCC cohort (by targeted sequencing, >500×mean depth covering 92.2% of all 9 MAPK1 exons). No germline mutations are found. Importantly, among which, 2 patients bore primary-to-recurrence somatic MAPK1 mutations, namely MAPK1p.D321N and p.R135K mutations (FIG. 1a). For the HK-T015 patient, his recurrent tumor carried an apparent increase of the MAPK1p.D321N allele frequency from 18.27% (primary) to 39.14% (recurrent), indicative of a likely driver activity during recurrence. Whereas the allele frequency of MAPK1p.R135K from primary to recurrent tumor did not change in the other patient, HK-T014.

### Residues E322, D321, and R135 in 3D Proximity

The present inventors mapped all MAPK1 somatic mutations from pan-cancers⁹⁻¹¹ and identified MAPK1 hotspot mutation cluster regions (arbitrarily defined in this study as mutation sites with >5 mutations) at amino acid residues E322 and D321, followed by the lesser frequent mutation cluster regions at E81, R135, R148 and S246 of the MAPK1 (ERK2) protein (FIG. 1b). D321 resides on the same DEP conserved sequence as E322, which is located right near the highly conserved kinase-interaction motif (KIM) of MAPK1 across species (FIG. 1c). KIM-docking domain is a conserved functional domain among all MAPKs known to be involved in kinase interactions¹². To further understand the potential impact of HNSCC-associated MAPK1 hotspot mutations (p.E322K, p.D321N, p.R135K) in relation to the ERK2 protein structure, the 3D locations of residues E322, D321 and R135 were mapped on the resolved x-ray crystallography structure of the human MAPK1(ERK2) (the structure was resolved with an ATP competitive inhibitor 5-Iodotubercidin and the allosteric inhibitor peptide-type ERK2 inhibitor; PDB ID: 5AX3¹³; MMDB ID: 136379¹⁴). Strikingly, all 3 residues cluster in close 3D proximity of only 9.0 Å to 12.8 Å from each other (but distant from the ATP binding site), and all are located on the “exposed” surface of ERK2 and belong to the KIM-docking domain of MAPK1, indicating that mutations of these residues potentially affect MAPK1's protein interactions with other kinases (FIGS. 1d & e).

### MAPK1p.D321N & p.R135K Drive EGFR Activation

Next, the potential driver activity of these 2 mutations was examined in vitro. Ectopic expression of MAPK1p.D321N and p.R135K in FaDu cells revealed opposite driver activities of these mutations. MAPK1p.D321N was a driver for HNSCC cell growth (22.3% growth increase vs MAPK1-WT; P<0.0001), while MAPK1p.R135K was unexpectedly a moderate suppressor for HNSCC cell growth (11.4% growth inhibition vs MAPK1 wild-type (WT), P<0.0001) (Figure if). Besides differences in their driver activities, these two mutations also demonstrated differential ability to activate EGFR. Ectopic expressions of MAPK1p.D321N in FaDu resulted in 2.14-fold increase in EGFR(Y1173) phosphorylation, a well-known EGFR transphosphorylation site for its activation, while a relatively lower level of EGFR activation was noted with MAPK1p.R135K (FIG. 1g, original uncropped image of the blots was shown in Supplementary FIG. 1). These findings on MAPK1p.D321N being a potent driver with high level of EGFR activation indicate functionally similarities between p.D321N and the previously reported p.E322K mutation, which caused heightened sensitivity to erlotinib in the HNSCC exceptional responder⁶.

### MAPK1p.D321N is Erlotinib-Sensitive In Vivo

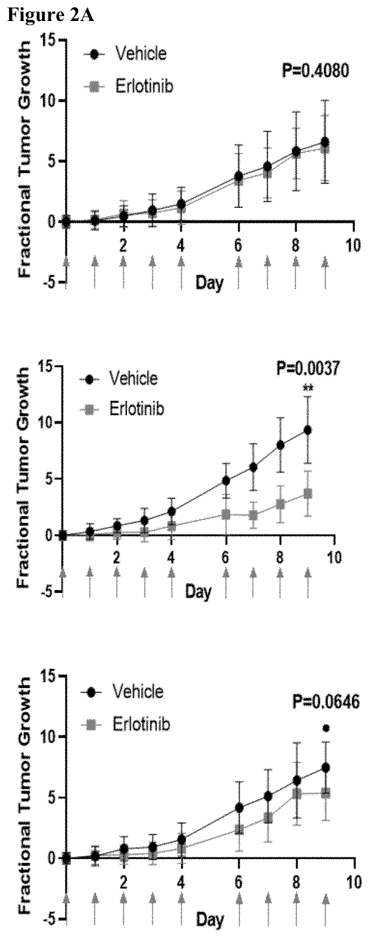

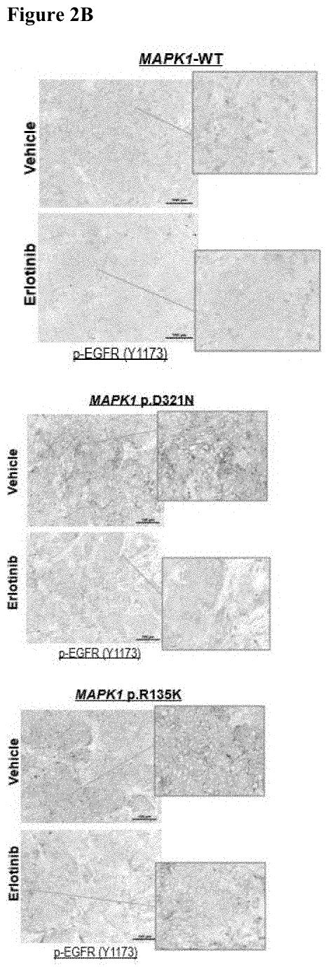

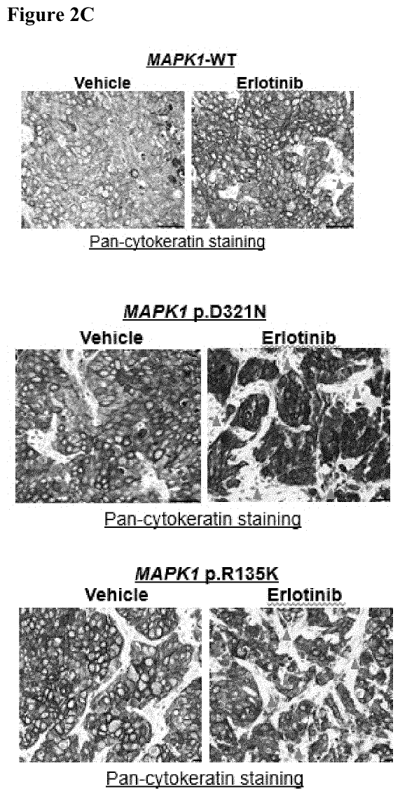

Prompted by their previous finding that MAPK1p.E322K could demonstrate heightened sensitivity to erlotinib in vivo reminiscent of the patient's clinical response in the exceptional responder, the inventors generated isogenic tumor xenografts expressing MAPK1-WT, MAPK1p.D321N and MAPK1p.R135K and compared their in vivo erlotinib responses. As shown by FIG. 2b, xenografts of the MAPK1p.D321N and p.R135K mutants both showed 80-90% cells with membranous staining of p-EGFR demonstrative of activated EGFR, while only 1% positivity was noted in the MAPK1-WT xenografts, which was consistent with the in vitro findings that both mutants were capable of activating EGFR (FIG. 1g). For mice bearing FaDu-MAPK1 WT xenografts, erlotinib treatment did not result in any change in tumor size or membranous p-EGFR signal in the tumors (vs. vehicle control, P=0.4080; FIG. 2a-b). Importantly, for MAPK1p.D321N mutant tumors, erlotinib treatment resulted in a significant reduction in tumor volume (60.3% reduction vs. vehicle treatment, P=0.0037; FIG. 2a), with concomitant increases in tumor-negative areas (i.e. cytokeratin-negative; FIG. 2c), as well as dramatic decreases in activated p-EGFR (membranous) in the tumors with less than 20% of membranous p-EGFR signals remaining (FIG. 2b). Of note, among MAPK1p.R135K mutant xenografts, erlotinib treatment caused a trend for tumor size reduction (28.0% reduction vs. vehicle treatment; P=0.0646; FIG. 2a), with noticeable increases in tumor-negative areas (FIG. 2c, cytokeratin-negative area). Notably, a moderate reduction of percent tumor cells bearing membranous p-EGFR staining was observed (50% vs. 90% in the vehicle treated MAPK1p.R135K mutant tumors, FIG. 2b). Thus, this study demonstrated a heightened sensitivity of MAPK1p.D321N to erlotinib in vivo, reminiscent of p.E322K reported previously⁶, providing direct evidences for MAPK1 mutant-driven erlotinib sensitivity by both MAPK1p.D321N and p.E322K in HNSCC. The findings from this functional-drug annotation study, together with previous erlotinib exceptional response associated with MAPK1p.E322K provide multiple levels of evidences supporting MAPK1-based precision clinical trials using erlotinib in HNSCC settings. Based on the TCGA-HNSCC cohort (N=510)^{9,10}, and the Johns-Hopkins HNSCC cohort (N=32)¹⁵, the mutation rates of these two erlotinib-sensitive mutations range from 1.6-3.1% in HNSCC, which may account up to ˜10,980-21,975 HNSCC patients per year (based on 0.7 million new cases of HNSCC per year in 2018^{9,10,15}). The current finding on erlotinib-sensitivity of MAPK1p.D321N, together with the MAPK1p.E322K mutation in an erlotinib exceptional responder provide evidences for further investigations of these two mutations in clinical settings in HNSCC.

### DISCUSSION

Despite extensive genomic characterization of HNSCC, precision medicine indications for this aggressive cancer remain limited. The only precision medicine choice for HNSCC follows that of FDA pan-cancer approval of larotrectinib for any solid tumors with NTRK fusions, which is anticipated to be most relevant for salivary gland tumors among all HNSCC¹⁶. As of today, though EGFR targeted therapy has been approved for HNSCC since 2006, the actual “precision way” of using EGFR inhibitors for HNSCC remains poorly defined.

In previously reported findings with the first exceptional responder of HNSCC for EGFR inhibitor, the patient's tumor harbored MAPK1p.E322K mutation, which was then subsequently proven to confer heightened sensitivity to erlotinib in vivo^{6,7}. Here, 105 HNSCC tumors from Hong Kong were sequenced, and two other MAPK1 mutations: p.R135K and p.D321N, were identified in recurrent HNSCC patients (both have AJCC Stage T4a diseases with disease recurrences). More importantly, functional analyses demonstrated that both mutations upregulated p-EGFR (Y1173) in vitro and in vivo, as compared to MAPK1-WT. Specifically, MAPK1p.D321N mutation, which caused a high level of EGFR activation in HNSCC cells, conferred significant sensitivity to erlotinib in vivo, concordant with the exceptional responder report for MAPK1p.E322K mutation^{6,7}. Such demonstrated functional similarities between MAPK1p.D321N mutation and MAPK1p.E322K (in the erlotinib exceptional responder^{6,7}), in terms of driver activities, EGFR hyperactivating capabilities, and erlotinib sensitivities, do provide direct evidence for potential use of the EGFR inhibitor, erlotinib, for MAPK1p.D321N and p.E322K-mutated HNSCC in a precision manner. Further clinical trial is warranted for translating these findings to clinical utility. Lastly, it is worth-investigating if tumors of other cancer types bearing MAPK1p.D321N and MAPK1p.E322K mutations are also erlotinib-sensitizing or not. These may include cervical cancer, bladder cancer and lung SCC based on our pan-cancer MAPK1 mutation mapping (FIG. 1b). Estimation for HNSCC alone, ˜10,980-21,975 HNSCC patients/year may bear these two erlotinib-sensitive mutations with potential therapeutic benefits (based on a 1.6-3.1% mutation rate and 0.7 million new HNSCC cases per year in 2018^{9,10,15})

### Methods

Tumor samples and Targeted Sequencing: MAPK1 targeted sequencing was analyzed using the IonS5 platform and Ion Reporter (ThermoFisher Scientific, USA). Clinical ethics approvals were obtained from the Research Ethics Committee of the Hospital Authority (University of Hong Kong/Hong Kong East Cluster; Joint Chinese University of Hong Kong-New Territories East Cluster; Kowloon West Cluster), Hong Kong SAR.

Retroviral vectors and Infection: pMXs-puro-MAPK1-WT, pMXs-puro-MAPK1 p.D321N and pMXs-puro-MAPK1 p.R135K were generated by site-directed mutagenesis with Sanger sequencing confirmation. Vectors were transfected into Plat-A retroviral production cells (Cell Biolabs, USA) for 3 days, and retroviruses were collected and used for infection for FaDu cells (purchased from ATCC, USA) as previously published⁶. Expression of MAPK1 mutants were confirmed by Western blotting. Infected cells were plated at 1.2×10⁵ cells/well in 48 well plate, and subjected to 5% FBS growth conditions for 96 hrs. MTT assays were then performed to determine the driver activity for growth vs EGFP-control. Cumulative results from three independent experiments with a total N≥14 wells were plotted.

Western blotting: Cell lysates were collected and analyzed by 8% SDS-PAGE, followed by primary antibody and secondary antibody incubations, and subsequent chemiluminescence development as previously¹⁷. Antibodies for p-EGFR (Y1173) is from Abcam UK (Cat: ab32578, 1:1000), total EGFR (Cat. 4267, 1:1000), p-MAPK(T202/Y204) (Cat: 9101, 1:2000), total MAPK (Cat. 9102, 1:2000) were purchased from Cell Signaling Technologies, USA. β-Actin antibody (Cat. sc-69879, 1:3000) was purchased from Santa Cruz, USA. GOAT X RABBIT-HRP (Bio-rad, cat: 170-6515, 1:2000) or GOAT X MOUSE-HRP (Bio-rad, cat: 170-6516, 1:2000) were used for secondary antibody incubation depending on the source of primary antibodies. All blots were derived from the same experiment and were processed in parallel.

Immunohistochemistry (IHC): IHC was performed as previously described¹⁸. The VECTASTAIN Elite ABC Universal PLUS Kit Peroxidase (Horse Anti-Mouse/Rabbit IgG) (Cat: PKK-8200) was used for IHC. Cytokeratin mouse antibody (DAKO, Cat: M3515, 1:500) and Anti-EGFR (phosphor Y1173) antibody [E124] (Abcam, Cat: ab32578, 1:100) were used as primary antibodies.

In vivo experiments: All animal experiments were approved by the University Animal Experimentation Ethics Committee of the Chinese University of Hong Kong. Isogenic FaDu cells were infected with MAPK1-WT and MAPK1 mutants by retrovirus and injected into nude mice subcutaneously (8×10⁵ cell per mouse, age of 4-5 weeks). Mice injected with respective infected FaDu cells were randomized into Erlotinib treatment group or Vehicle group (2 tumors born by each mice, 4 mice per group). Treatment started at day 6 after injection with 6 dose per week until day 15. Erlotinib (dissolved in 10% HP-j-CD) or vehicle control were administered orally at a dose of 50 mg/kg. Tumor volume was monitored and calculated by the equation: length×width²/2 repeatedly for 5 consecutive days as indicated by the arrows on the X-axis of FIG. 2(a). The mice were sacrifice at the end-point of the experiment.

Statistical analysis: Student t-test (with nonparametric Mann-Whitney test, two-sided) were performed using the GraphPad Prism software.

### Code Availability

No computer code was used in this study.

### Data Availability

The data/reanalysis that support the findings of this study are publicly available online at the following websites: cbioportal.org; cancer.sanger.ac.uk/cosmic; and ncbi.nlm.nih.gov/Structure/index.shtml. All other data supporting the findings of this study are available upon request.

All patents, patent applications, and other publications, including GenBank Accession Numbers, cited in this application are incorporated by reference in the entirety for all purposes.

### LIST OF REFERENCES

1 Zhou, C. et al. Erlotinib versus chemotherapy as first-line treatment for patients with advanced EGFR mutation-positive non-small-cell lung cancer (OPTIMAL, CTONG-0802): a multicentre, open-label, randomised, phase 3 study. Lancet Oncol 12, 735-742, doi:10.1016/S1470-2045(11)70184-X (2011).
2 Rosell, R. et al. Erlotinib versus standard chemotherapy as first-line treatment for European patients with advanced EGFR mutation-positive non-small-cell lung cancer (EURTAC): a multicentre, open-label, randomised phase 3 trial. Lancet Oncol 13, 239-246, doi:10.1016/S1470-2045(11)70393-X (2012).
3 Li, Z. et al. Erlotinib effectively inhibits JAK2V617F activity and polycythemia vera cell growth. J Biol Chem 282, 3428-3432, doi:10.1074/jbc.C600277200 (2007).
4 Tan, D. S. W. et al. Long noncoding RNA EGFR-AS1 mediates epidermal growth factor receptor addiction and modulates treatment response in squamous cell carcinoma. Nat Med 23, 1167-1175, doi:10.1038/nm.4401 (2017).
Bauman, J. E. et al. Randomized, placebo-controlled window trial of EGFR, Src, or combined blockade in head and neck cancer. JCI Insight 2, e90449, doi:10.1172/jci.insight.90449 (2017).
6 Van Allen, E. M. et al. Genomic Correlate of Exceptional Erlotinib Response in Head and Neck Squamous Cell Carcinoma. JAMA Oncol 1, 238-244, doi:10.1001/jamaoncol.2015.34 (2015).
7 Wen, Y. et al. MAPK1E322K mutation increases head and neck squamous cell carcinoma sensitivity to erlotinib through enhanced secretion of amphiregulin. Oncotarget 7, 23300-23311, doi:10.18632/oncotarget.8188 (2016).
8 Lala, M., Chirovsky, D., Cheng, J. D. & Mayawala, K. Clinical outcomes with therapies for previously treated recurrent/metastatic head-and-neck squamous cell carcinoma (R/M HNSCC): A systematic literature review. Oral Oncol 84, 108-120, doi:10.1016/j.oraloncology.2018.07.005 (2018).
9 Cerami, E. et al. The cBio cancer genomics portal: an open platform for exploring multidimensional cancer genomics data. Cancer Discov 2, 401-404, doi:10.1158/2159-8290.CD-12-0095 (2012).
Gao, J. et al. Integrative analysis of complex cancer genomics and clinical profiles using the cBioPortal. Sci Signal 6, pl1, doi:10.1126/scisignal.2004088 (2013).
11 Tate, J. G. et al. COSMIC: the Catalogue Of Somatic Mutations In Cancer. Nucleic Acids Res 47, D941-D947, doi:10.1093/nar/gky1015 (2019).
12 Liu, S., Sun, J. P., Zhou, B. & Zhang, Z. Y. Structural basis of docking interactions between ERK2 and MAP kinase phosphatase 3. Proc Natl Acad Sci USA 103, 5326-5331, doi:10.1073/pnas.0510506103 (2006).
13 Kinoshita, T., Sugiyama, H., Mori, Y., Takahashi, N. & Tomonaga, A. Identification of allosteric ERK2 inhibitors through in silico biased screening and competitive binding assay. Bioorg Med Chem Lett 26, 955-958, doi:10.1016/j.bmcl.2015.12.056 (2016).
14 Madej, T. et al. MMDB and VAST+: tracking structural similarities between macromolecular complexes. Nucleic Acids Res 42, D297-303, doi:10.1093/nar/gkt1208 (2014).
Agrawal, N. et al. Exome sequencing of head and neck squamous cell carcinoma reveals inactivating mutations in NOTCH1. Science 333, 1154-1157, doi:10.1126/science.1206923 (2011).
16 Skalova, A. et al. Mammary analogue secretory carcinoma of salivary glands, containing the ETV6-NTRK3 fusion gene: a hitherto undescribed salivary gland tumor entity. Am J Surg Pathol 34, 599-608, doi:10.1097/PAS.0b013e3181d9efcc (2010).
17 Lui, V. W. et al. Frequent mutation of the PI3K pathway in head and neck cancer defines predictive biomarkers. Cancer Discov 3, 761-769, doi:10.1158/2159-8290.CD-13-01032159-8290.CD-13-0103 [pii] (2013).
18 Li, Y. Y. et al. Exome and genome sequencing of nasopharynx cancer identifies NF-kappaB pathway activating mutations. Nat Commun 8, 14121, doi:10.1038/ncomms14121 (2017).

| Sequence Listing | |
|---|---|
| Human MAPK1 protein amino acid sequence (GenBank Accession No. | |
| NP_002736.3; KIM domain amino acid residues 81, 109-110, 114-115, | |
| 118-119, -125, 128, 131, 135, 157-162, 316, 318, and 321 are | |
| underlined) | |
| SEQ ID NO: 1 | |
| 10 20 30 40 50 | |
| MAAAAAAGAG PEMVRGQVFD VGPRYTNLSY IGEGAYGMVC SAYDNVNKVR | |
| 60 70 80 90 100 | |
| VAIKKISPFE HQTYCQRTLR EIKILLRFRH ENIIGINDII RAPTIEQMKD | |
| 110 120 130 140 150 | |
| VYIVQDLMET DLYKLLKTQH LSNDHICYFL YQILRGLKYI HSANVLHRDL | |
| 160 170 180 190 200 | |
| KPSNLLLNTT CDLKICDFGL ARVADPDHDH TGFLTEYVAT RWYRAPEIML | |
| 210 220 230 240 250 | |
| NSKGYTKSID IWSVGCILAE MLSNRPIFPG KHYLDQLNHI LGILGSPSQE | |
| 260 270 280 290 300 | |
| DLNCIINLKA RNYLLSLPHK NKVPWNRLFP NADSKALDLL DKMLTFNPHK | |
| 310 320 330 340 350 | |
| RIEVEQALAH PYLEQYYDPS DEPIAEAPFK FDMELDDLPK EKLKELIFEE | |
| 360 | |
| TARFQPGYRS | |
| Human MAPK1 genomic DNA sequence (GenBank Accession No. | |
| NM_002745.5) | |
| SEQ ID NO: 2 | |
| 1 | agtctggcag gcaggcaggc aatcggtccg agtggctgtc ggctcttcag ctctcccgct |
| 61 | cggcgtcttc cttcctcctc ccggtcagcg tcggcggctg caccggcggc ggcgcagtcc |
| 121 | ctgcgggagg ggcgacaaga gctgagcggc ggccgccgag cgtcgagctc agcgcggcgg |
| 181 | aggcggcggc ggcccggcag ccaacatggc ggcggcggcg gcggcgggcg cgggcccgga |
| 241 | gatggtccgc gggcaggtgt tcgacgtggg gccgcgctac accaacctct cgtacatcgg |
| 301 | cgagggcgcc tacggcatgg tgtgctctgc ttatgataat gtcaacaaag ttcgagtagc |
| 361 | tatcaagaaa atcagcccct ttgagcacca gacctactgc cagagaaccc tgagggagat |
| 421 | aaaaatctta ctgcgcttca gacatgagaa catcattgga atcaatgaca ttattcgagc |
| 481 | accaaccatc gagcaaatga aagatgtata tatagtacag gacctcatgg aaacagatct |
| 541 | ttacaagctc ttgaagacac aacacctcag caatgaccat atctgctatt ttctctacca |
| 601 | gatcctcaga gggttaaaat atatccattc agctaacgtt ctgcaccgtg acctcaagcc |
| 661 | ttccaacctg ctgctcaaca ccacctgtga tctcaagatc tgtgactttg gcctggcccg |
| 721 | tgttgcagat ccagaccatg atcacacagg gttcctgaca gaatatgtgg ccacacgttg |
| 781 | gtacagggct ccagaaatta tgttgaattc caagggctac accaagtcca ttgatatttg |
| 841 | gtctgtaggc tgcattctgg cagaaatgct ttctaacagg cccatctttc cagggaagca |
| 901 | ttatcttgac cagctgaacc acattttggg tattcttgga tccccatcac aagaagacct |
| 961 | gaattgtata ataaatttaa aagctaggaa ctatttgctt tctcttccac acaaaaataa |
| 1021 | ggtgccatgg aacaggctgt tcccaaatgc tgactccaaa gctctggact tattggacaa |
| 1081 | aatgttgaca ttcaacccac acaagaggat tgaagtagaa caggctctgg cccacccata |
| 1141 | tctggagcag tattacgacc cgagtgacga gcccatcgcc gaagcaccat tcaagttcga |
| 1201 | catggaattg gatgacttgc ctaaggaaaa gctcaaagaa ctaatttttg aagagactgc |
| 1261 | tagattccag ccaggataca gatcttaaat ttgtcaggac aagggctcag aggactggac |
| 1321 | gtgctcagac atcggtgttc ttcttcccag ttcttgaccc ctggtcctgt ctccagcccg |
| 1381 | tcttggctta tccactttga ctcctttgag ccgtttggag gggcggtttc tggtagttgt |
| 1441 | ggcttttatg ctttcaaaga atttcttcag tccagagaat tcctcctggc agccctgtgt |
| 1501 | gtgtcaccca ttggtgacct gcggcagtat gtacttcagt gcacctactg cttactgttg |
| 1561 | ctttagtcac taattgcttt ctggtttgaa agatgcagtg gttcctccct ctcctgaatc |
| 1621 | cttttctaca tgatgccctg ctgaccatgc agccgcacca gagagagatt cttccccaat |
| 1681 | tggctctagt cactggcatc tcactttatg atagggaagg ctactaccta gggcacttta |
| 1741 | agtcagtgac agccccttat ttgcacttca ccttttgacc ataactgttt ccccagagca |
| 1801 | ggagcttgtg gaaatacctt ggctgatgtt gcagcctgca gcaagtgctt ccgtctccgg |
| 1861 | aatccttggg gagcacttgt ccacgtcttt tctcatatca tggtagtcac taacatatat |
| 1921 | aaggtatgtg ctattggccc agcttttaga aaatgcagtc atttttctaa ataaaaagga |
| 1981 | agtactgcac ccagcagtgt cactctgtag ttactgtggt cacttgtacc atatagaggt |
| 2041 | gtaacacttg tcaagaagcg ttatgtgcag tacttaatgt ttgtaagact tacaaaaaaa |
| 2101 | gatttaaagt ggcagcttca ctcgacattt ggtgagagaa gtacaaaggt tgcagtgctg |
| 2161 | agctgtgggc ggtttctggg gatgtcccag ggtggaactc cacatgctgg tgcatatacg |
| 2221 | cccttgagct acttcaaatg tgggtgtttc agtaaccacg ttccatgcct gaggatttag |
| 2281 | cagagaggaa cactgcgtct ttaaatgaga aagtatacaa ttctttttcc ttctacagca |
| 2341 | tgtcagcatc tcaagttcat ttttcaacct acagtataac aatttgtaat aaagcctcca |
| 2401 | ggagctcatg acgtgaagca ctgttctgtc ctcaagtact caaatatttc tgatactgct |
| 2461 | gagtcagact gtcagaaaaa gctagcacta actcgtgttt ggagctctat ccatatttta |
| 2521 | ctgatctctt taagtatttg ttcctgccac tgtgtactgt ggagttgact cggtgttctg |
| 2581 | tcccagtgcg gtgcctcctc ttgacttccc cactgctctc tgtggtgaga aatttgcctt |
| 2641 | gttcaataat tactgtaccc tcgcatgact gttacagctt tctgtgcaga gatgactgtc |
| 2701 | caagtgccac atgcctacga ttgaaatgaa aactctattg ttacctctga gttgtgttcc |
| 2761 | acggaaaatg ctatccagca gatcatttag gaaaaataat tctattttta gcttttcatt |
| 2821 | tctcagctgt ccttttttct tgtttgattt ttgacagcaa tggagaatgg gttatataaa |
| 2881 | gactgcctgc taatatgaac agaaatgcat ttgtaattca tgaaaataaa tgtacatctt |
| 2941 | ctatcttcac attcatgtta agattcagtg ttgctttcct ctggatcagc gtgtctgaat |
| 3001 | ggacagtcag gttcaggttg tgctgaacac agaaatgctc acaggcctca ctttgccgcc |
| 3061 | caggcactgg cccagcactt ggatttacat aagatgagtt agaaaggtac ttctgtaggg |
| 3121 | tcctttttac ctctgctcgg cagagaatcg atgctgtcat gttcctttat tcacaatctt |
| 3181 | aggtctcaaa tattctgtca aaccctaaca aagaagcccc gacatctcag gttggattcc |
| 3241 | ctggttctct ctaaagaggg cctgcccttg tgccccagag gtgctgctgg gcacagccaa |
| 3301 | gagttgggaa gggccgcccc acagtacgca gtcctcacca cccagcccag ggtgctcacg |
| 3361 | ctcaccactc ctgtggctga ggaaggatag ctggctcatc ctcggaaaac agacccacat |
| 3421 | ctctattctt gccctgaaat acgcgctttt cacttgcgtg ctcagagctg ccgtctgaag |
| 3481 | gtccacacag cattgacggg acacagaaat gtgactgtta ccggataaca ctgattagtc |
| 3541 | agttttcatt tataaaaaag cattgacagt tttattactc ttgtttcttt ttaaatggaa |
| 3601 | agttactatt ataaggttaa tttggagtcc tcttctaaat agaaaaccat atccttggct |
| 3661 | actaacatct ggagactgtg agctccttcc cattcccctt cctggtactg tggagtcaga |
| 3721 | ttggcatgaa accactaact tcattctaga atcattgtag ccataagttg tgtgcttttt |
| 3781 | attaatcatg ccaaacataa tgtaactggg cagagaatgg tcctaaccaa ggtacctatg |
| 3841 | aaaagcgcta gctatcatgt gtagtagatg catcattttg gctcttctta catttgtaaa |
| 3901 | aatgtacaga ttaggtcatc ttaattcata ttagtgacac ggaacagcac ctccactatt |
| 3961 | tgtatgttca aataagcttt cagactaata gcttttttgg tgtctaaaat gtaagcaaaa |
| 4021 | aattcctgct gaaacattcc agtcctttca tttagtataa aagaaatact gaacaagcca |
| 4081 | gtgggatgga attgaaagaa ctaatcatga ggactctgtc ctgacacagg tcctcaaagc |
| 4141 | tagcagagat acgcagacat tgtggcatct gggtagaaga atactgtatt gtgtgtgcag |
| 4201 | tgcacagtgt gtggtgtgtg cacactcatt ccttctgctc ttgggcacag gcagtgggtg |
| 4261 | tagaggtaac cagtagcttt gagaagctac atgtagctca ccagtggttt tctctaagga |
| 4321 | atcacaaaag taaactaccc aaccacatgc cacgtaatat ttcagccatt cagaggaaac |
| 4381 | tgttttctct ttatttgctt atatgttaat atggttttta aattggtaac ttttatatag |
| 4441 | tatggtaaca gtatgttaat acacacatac atacgcacac atgctttggg tccttccata |
| 4501 | atacttttat atttgtaaat caatgttttg gagcaatccc aagtttaagg gaaatatttt |
| 4561 | tgtaaatgta atggttttga aaatctgagc aatccttttg cttatacatt tttaaagcat |
| 4621 | ttgtgcttta aaattgttat gctggtgttt gaaacatgat actcctgtgg tgcagatgag |
| 4681 | aagctataac agtgaatatg tggtttctct tacgtcatcc accttgacat gatgggtcag |
| 4741 | aaacaaatgg aaatccagag caagtcctcc agggttgcac caggtttacc taaagcttgt |
| 4801 | tgccttttct tgtgctgttt atgcgtgtag agcactcaag aaagttctga aactgctttg |
| 4861 | tatctgcttt gtactgttgg tgccttcttg gtattgtacc ccaaaattct gcatagatta |
| 4921 | tttagtataa tggtaagtta aaaaatgtta aaggaagatt ttattaagaa tctgaatgtt |
| 4981 | tattcattat attgttacaa tttaacatta acatttattt gtggtatttg tgatttggtt |
| 5041 | aatctgtata aaaattgtaa gtagaaaggt ttatatttca tcttaattct tttgatgttg |
| 5101 | taaacgtact ttttaaaaga tggattattt gaatgtttat ggcacctgac ttgtaaaaaa |
| 5161 | aaaaaactac aaaaaaatcc ttagaatcat taaattgtgt ccctgtatta ccaaaataac |
| 5221 | acagcaccgt gcatgtatag tttaattgca gtttcatctg tgaaaacgtg aaattgtcta |
| 5281 | gtccttcgtt atgttcccca gatgtcttcc agatttgctc tgcatgtggt aacttgtgtt |
| 5341 | agggctgtga gctgttcctc gagttgaatg gggatgtcag tgctcctagg gttctccagg |
| 5401 | tggttcttca gaccttcacc tgtggggggg ggggtaggcg gtgcccacgc ccatctcctc |
| 5461 | atcctcctga acttctgcaa ccccactgct gggcagacat cctgggcaac ccctttttc |
| 5521 | agagcaagaa gtcataaaga taggatttct tggacatttg gttcttatca atattgggca |
| 5581 | ttatgtaatg acttatttac aaaacaaaga tactggaaaa tgttttggat gtggtgttat |
| 5641 | ggaaagagca caggccttgg acccatccag ctgggttcag aactaccccc tgcttataac |
| 5701 | tgcggctggc tgtgggccag tcattctgcg tctctgcttt cttcctctgc ttcagactgt |
| 5761 | cagctgtaaa gtggaagcaa tattacttgc cttgtatatg gtaaagatta taaaaataca |
| 5821 | tttcaactgt tcagcatagt acttcaaagc aagtactcag taaatagcaa gtctttttaa |
| 5881 | a |

1. A method for assessing likelihood of effective anti-EGFR cancer therapy in a cancer patient, comprising the steps of:
(a) obtaining genomic DNA from a biological sample taken from the patient;
(b) determining nucleotide sequence of a portion of MAPK1 genomic sequence encoding at least one of the 77-84, 131-139, 143-152, 241-250, and 317-325 segments of SEQ ID NO:1;
(c) detecting one or more mutations within the portion of MAPK1 genomic sequence encoding at least one of the 77-84, 131-139, 143-152, 241-250, and 317-325 segments of SEQ ID NO:1; and
(d) determining the cancer patient as likely to achieve effective anti-EGFR cancer therapy.

2. The method of claim 1, wherein the biological sample is a cancer biopsy or a blood sample.

3. The method of claim 1, wherein the cancer is head and neck squamous cell carcinoma (HNSCC).

4. The method of claim 1, wherein step (b) comprises determining nucleotide sequence of the portion of MAPK1 genomic sequence encoding at least one of the 81-84, 135-139, and 317-321 segments of SEQ ID NO: 1.

5. The method of claim 1, wherein step (c) comprises detecting one or more mutations at residue 81, 135, or 321 of SEQ ID NO:1.

6. The method of claim 5, wherein the one or more mutations comprise at least one substitution at residue 81, 135, or 321 of SEQ 1ID NO:1.

7. The method of claim 6, wherein the one or more mutations comprise R135K or D321N in SEQ ID NO:1.

8. The method of claim 1, wherein step (c) comprises detecting one or more mutations at residue 148 or 246 of SEQ ID NO:1.

9. The method of claim 1, wherein step (b) further comprises determining nucleotide sequence of the portion of MAPK1 genomic sequence encoding a segment of SEQ ID NO:1 encompassing residue 322.

10. The method of claim 1, further comprising, subsequent to step (d), administering to the cancer patient an EGFR inhibitor.

11. The method of claim 10, wherein the EGFR inhibitor comprises erlotinib.

12. The method of claim 1, wherein step (b) comprises a polymerase chain reaction (PCR).

13. The method of claim 1, wherein step (b) comprises a polynucleotide sequencing reaction or a polynucleotide hybridization assay.

14. A kit for assessing likelihood of effective anti-EGFR cancer therapy in a cancer patient, comprising (1) two oligonucleotide primers capable of specifically amplifying a portion of MAPK1 genomic sequence obtained from a biological sample taken from the cancer patient, wherein the portion of MAPK1 genomic sequence encodes at least one of the 77-84, 131-139, 143-152, 241-250, and 317-325 segments of SEQ ID NO:1; and (2) an agent capable of determining nucleotide sequence of the portion of MAPK1 genomic sequence encoding at least one of the 77-84, 131-139, 143-152, 241-250, and 317-325 segments of SEQ ID NO:1.

15-18. (canceled)

19. A method for treating a cancer patient, comprising the step of:
(i) administering to a cancer patient whose genomic sequence comprises at least one mutation in a portion of MAPK1 genomic sequence encoding at least one of the 77-84, 131-139, 143-152, 241-250, and 317-325 segments of SEQ ID NO:1 an effective amount of an EGFR inhibitor.

20. The method of claim 19, comprising, prior to step (i), a step of:
selecting a cancer patient whose genomic sequence has been analyzed and confirmed to comprise at least one mutation in a portion of MAPK1 genomic sequence encoding at least one of the 77-84, 131-139, 143-152, 241-250, and 317-325 segments of SEQ ID NO:1.

21. The method of claim 19, wherein the patient has at least one mutation in the portion of MAPK1 genomic sequence encoding at least one of the 77-84, 135-139, and 317-325 segments of SEQ ID NO:1.

22. The method of claim 19, wherein the patient has at least one mutation at residues 81, 135, and 321 of SEQ ID NO:1.

23. The method of claim 22, wherein the patient has at least one substitution at residues 81, 135, and 321 of SEQ ID NO:1.

24. The method of claim 23, wherein the patient has at least one mutation of R135K or D321N in SEQ ID NO 1.

25-27. (canceled)


SEQUENCE LISTING (22 sequences)

SEQ ID NO: 1 - protein, 360 aa, Homo sapiens [sequence omitted]

SEQ ID NO: 2 - DNA, 5881 bp, Homo sapiens
agtctggcaggcaggcaggcaatcggtccgagtggctgtcggctcttcagctctcccgctcggcgtcttccttcctcctcccggtcagcgtcggcggctgcaccggcggcggcgcagtccctgcgggaggggcgacaagagctgagcggcggccgccgagcgtcgagctcagcgcggcggaggcggcggcggcccggcagccaacatggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagcattatcttgaccagctgaaccacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggatacagatcttaaatttgtcaggacaagggctcagaggactggacgtgctcagacatcggtgttcttcttcccagttcttgacccctggtcctgtctccagcccgtcttggcttatccactttgactcctttgagccgtttggaggggcggtttctggtagttgtggcttttatgctttcaaagaatttcttcagtccagagaattcctcctggcagccctgtgtgtgtcacccattggtgacctgcggcagtatgtacttcagtgcacctactgcttactgttgctttagtcactaattgctttctggtttgaaagatgcagtggttcctccctctcctgaatccttttctacatgatgccctgctgaccatgcagccgcaccagagagagattcttccccaattggctctagtcactggcatctcactttatgatagggaaggctactacctagggcactttaagtcagtgacagccccttatttgcacttcaccttttgaccataactgtttccccagagcaggagcttgtggaaataccttggctgatgttgcagcctgcagcaagtgcttccgtctccggaatccttggggagcacttgtccacgtcttttctcatatcatggtagtcactaacatatataaggtatgtgctattggcccagcttttagaaaatgcagtcatttttctaaataaaaaggaagtactgcacccagcagtgtcactctgtagttactgtggtcacttgtaccatatagaggtgtaacacttgtcaagaagcgttatgtgcagtacttaatgtttgtaagacttacaaaaaaagatttaaagtggcagcttcactcgacatttggtgagagaagtacaaaggttgcagtgctgagctgtgggcggtttctggggatgtcccagggtggaactccacatgctggtgcatatacgcccttgagctacttcaaatgtgggtgtttcagtaaccacgttccatgcctgaggatttagcagagaggaacactgcgtctttaaatgagaaagtatacaattctttttccttctacagcatgtcagcatctcaagttcatttttcaacctacagtataacaatttgtaataaagcctccaggagctcatgacgtgaagcactgttctgtcctcaagtactcaaatatttctgatactgctgagtcagactgtcagaaaaagctagcactaactcgtgtttggagctctatccatattttactgatctctttaagtatttgttcctgccactgtgtactgtggagttgactcggtgttctgtcccagtgcggtgcctcctcttgacttccccactgctctctgtggtgagaaatttgccttgttcaataattactgtaccctcgcatgactgttacagctttctgtgcagagatgactgtccaagtgccacatgcctacgattgaaatgaaaactctattgttacctctgagttgtgttccacggaaaatgctatccagcagatcatttaggaaaaataattctatttttagcttttcatttctcagctgtccttttttcttgtttgatttttgacagcaatggagaatgggttatataaagactgcctgctaatatgaacagaaatgcatttgtaattcatgaaaataaatgtacatcttctatcttcacattcatgttaagattcagtgttgctttcctctggatcagcgtgtctgaatggacagtcaggttcaggttgtgctgaacacagaaatgctcacaggcctcactttgccgcccaggcactggcccagcacttggatttacataagatgagttagaaaggtacttctgtagggtcctttttacctctgctcggcagagaatcgatgctgtcatgttcctttattcacaatcttaggtctcaaatattctgtcaaaccctaacaaagaagccccgacatctcaggttggattccctggttctctctaaagagggcctgcccttgtgccccagaggtgctgctgggcacagccaagagttgggaagggccgccccacagtacgcagtcctcaccacccagcccagggtgctcacgctcaccactcctgtggctgaggaaggatagctggctcatcctcggaaaacagacccacatctctattcttgccctgaaatacgcgcttttcacttgcgtgctcagagctgccgtctgaaggtccacacagcattgacgggacacagaaatgtgactgttaccggataacactgattagtcagttttcatttataaaaaagcattgacagttttattactcttgtttctttttaaatggaaagttactattataaggttaatttggagtcctcttctaaatagaaaaccatatccttggctactaacatctggagactgtgagctccttcccattccccttcctggtactgtggagtcagattggcatgaaaccactaacttcattctagaatcattgtagccataagttgtgtgctttttattaatcatgccaaacataatgtaactgggcagagaatggtcctaaccaaggtacctatgaaaagcgctagctatcatgtgtagtagatgcatcattttggctcttcttacatttgtaaaaatgtacagattaggtcatcttaattcatattagtgacacggaacagcacctccactatttgtatgttcaaataagctttcagactaatagcttttttggtgtctaaaatgtaagcaaaaaattcctgctgaaacattccagtcctttcatttagtataaaagaaatactgaacaagccagtgggatggaattgaaagaactaatcatgaggactctgtcctgacacaggtcctcaaagctagcagagatacgcagacattgtggcatctgggtagaagaatactgtattgtgtgtgcagtgcacagtgtgtggtgtgtgcacactcattccttctgctcttgggcacaggcagtgggtgtagaggtaaccagtagctttgagaagctacatgtagctcaccagtggttttctctaaggaatcacaaaagtaaactacccaaccacatgccacgtaatatttcagccattcagaggaaactgttttctctttatttgcttatatgttaatatggtttttaaattggtaacttttatatagtatggtaacagtatgttaatacacacatacatacgcacacatgctttgggtccttccataatacttttatatttgtaaatcaatgttttggagcaatcccaagtttaagggaaatatttttgtaaatgtaatggttttgaaaatctgagcaatccttttgcttatacatttttaaagcatttgtgctttaaaattgttatgctggtgtttgaaacatgatactcctgtggtgcagatgagaagctataacagtgaatatgtggtttctcttacgtcatccaccttgacatgatgggtcagaaacaaatggaaatccagagcaagtcctccagggttgcaccaggtttacctaaagcttgttgccttttcttgtgctgtttatgcgtgtagagcactcaagaaagttctgaaactgctttgtatctgctttgtactgttggtgccttcttggtattgtaccccaaaattctgcatagattatttagtataatggtaagttaaaaaatgttaaaggaagattttattaagaatctgaatgtttattcattatattgttacaatttaacattaacatttatttgtggtatttgtgatttggttaatctgtataaaaattgtaagtagaaaggtttatatttcatcttaattcttttgatgttgtaaacgtactttttaaaagatggattatttgaatgtttatggcacctgacttgtaaaaaaaaaaaactacaaaaaaatccttagaatcattaaattgtgtccctgtattaccaaaataacacagcaccgtgcatgtatagtttaattgcagtttcatctgtgaaaacgtgaaattgtctagtccttcgttatgttccccagatgtcttccagatttgctctgcatgtggtaacttgtgttagggctgtgagctgttcctcgagttgaatggggatgtcagtgctcctagggttctccaggtggttcttcagaccttcacctgtgggggggggggtaggcggtgcccacgcccatctcctcatcctcctgaacttctgcaaccccactgctgggcagacatcctgggcaaccccttttttcagagcaagaagtcataaagataggatttcttggacatttggttcttatcaatattgggcattatgtaatgacttatttacaaaacaaagatactggaaaatgttttggatgtggtgttatggaaagagcacaggccttggacccatccagctgggttcagaactaccccctgcttataactgcggctggctgtgggccagtcattctgcgtctctgctttcttcctctgcttcagactgtcagctgtaaagtggaagcaatattacttgccttgtatatggtaaagattataaaaatacatttcaactgttcagcatagtacttcaaagcaagtactcagtaaatagcaagtctttttaaa

SEQ ID NO: 3 - protein, 42 aa, Homo sapiens
LTFNPHKRIEVEQALAHPYLEQYYDPSDEPIAEAPFKFDMEL

SEQ ID NO: 4 - protein, 42 aa, Pan troglodytes; identical to SEQ ID NO: 3

SEQ ID NO: 5 - protein, 42 aa, Macaca mulatta; identical to SEQ ID NO: 3

SEQ ID NO: 6 - protein, 42 aa, Canis lupus; identical to SEQ ID NO: 3

SEQ ID NO: 7 - protein, 42 aa, Bos taurus
LTFNPHKRIEVEQALAHPYLEQYYDPSDEPVAEAPFKFDMEL

SEQ ID NO: 8 - protein, 42 aa, Mus musculus; identical to SEQ ID NO: 3

SEQ ID NO: 9 - protein, 42 aa, Rattus norvegicus; identical to SEQ ID NO: 3

SEQ ID NO: 10 - protein, 42 aa, Gallus gallus; identical to SEQ ID NO: 7

SEQ ID NO: 11 - protein, 42 aa, Xenopus tropicalis
LTFNPHKRIEVEEALAHPYLEQYYDPTDEPVAEAPFKFDMEL

SEQ ID NO: 12 - protein, 42 aa, Danio rerio
LTFNPHKRIPVEEALAHPYLEQYYDPGDEPVAEVPFRINMEN

SEQ ID NO: 13 - protein, 45 aa, Homo sapiens
QDLMETDLYKLLKTQHLSNDHICYFLYQILRGLKYIHSANVLHRD

SEQ ID NO: 14 - protein, 45 aa, Pan troglodytes; identical to SEQ ID NO: 13

SEQ ID NO: 15 - protein, 45 aa, Macaca mulatta; identical to SEQ ID NO: 13

SEQ ID NO: 16 - protein, 45 aa, Canis lupus; identical to SEQ ID NO: 13

SEQ ID NO: 17 - protein, 45 aa, Bos taurus; identical to SEQ ID NO: 13

SEQ ID NO: 18 - protein, 45 aa, Mus musculus; identical to SEQ ID NO: 13

SEQ ID NO: 19 - protein, 45 aa, Rattus norvegicus; identical to SEQ ID NO: 13

SEQ ID NO: 20 - protein, 45 aa, Gallus gallus; identical to SEQ ID NO: 13

SEQ ID NO: 21 - protein, 45 aa, Xenopus tropicalis; identical to SEQ ID NO: 13

SEQ ID NO: 22 - protein, 45 aa, Danio rerio
QCLMETDLYKLLKTQRLSNDHICYFLYQILRGLKYIHSANVLHRD